(19) United States
(12) Patent Application Publication
(10) Pub. No.: US 2022/0297288 A1
(43) Pub. Date: Sep. 22, 2022

(54) FREE-STANDING ARTIFICIAL MUSCLES CONTAINING POLYMERIC ACTUATORS

(71) Applicant: Board of Supervisors of Louisiana State University and Agricultural and Mechanical College, Baton Rouge, LA (US)

(72) Inventors: Guoqiang Li, Baton Rouge, LA (US); Sarah Li, Baton Rouge, LA (US); Jizhou Fan, Baton Rouge, LA (US); Xiaming Feng, Baton Rouge, LA (US)

(21) Appl. No.: 17/655,014

(22) Filed: Mar. 16, 2022

Related U.S. Application Data
(60) Provisional application No. 63/161,535, filed on Mar. 16, 2021.

(51) Int. Cl. B25J 9/10; F03G 7/06

(52) U.S. Cl. CPC B25J 9/1075; B25J 9/1085; F03G 7/0612

(57) ABSTRACT

In one aspect, the disclosure relates to free-standing artificial muscles having a polymeric core encased by an elastic spring. The polymeric core can be any two-way shape memory polymer including, but not limited to, a semicrystalline polymer (polybutadiene polymer, a polycaprolactone polymer, a poly(ethylene-co-vinyl acetate)), a rubber, an ionomer, an elastomer, or a gel. In some aspects, the shape memory polymers are crosslinked. In an alternative aspect, the polymeric core is a twisted and coiled polymeric fiber. In other aspects, the polymeric core is reprocessable, remoldable, and/or recyclable. In one aspect, the elastic spring is metallic, ceramic, plastic, or any combination thereof. The stiffnesses or spring rate of the elastic spring and polymeric core, the two-way shape memory effect of the polymeric core, and their geometrical dimensions can be optimized to maximize the actuation strain based on theoretical principles described herein. Also disclosed are devices incorporating the free-standing artificial muscles.

### CROSS REFERENCE TO RELATED APPLICATIONS

This application claims the benefit of U.S. Provisional Application No. 63/161,535 filed on Mar. 16, 2021, which is incorporated herein by reference in its entirety.

### STATEMENT REGARDING FEDERALLY SPONSORED RESEARCH OR DEVELOPMENT

This invention was made with government support under grant number 1736136 awarded by the National Science Foundation. The government has certain rights in the invention.

### BACKGROUND

Artificial muscles, which contract upon heating and expand upon cooling, have found applications in many high tech fields such as soft robots, aeronautics, and medical devices. Previously, scientists have constructed artificial muscles out of polymers with negative coefficients of thermal expansion, such as nylon or polyethylene, or two-way shape memory polymers such as poly(ethylene-co-vinyl acetate). However, these muscles have shown to be impractical for actuation in soft robots and other related applications because a weight must be hung from the muscle for it to actually function. Furthermore, conventional metallic springs, which have been used widely, do not have muscle action.

Artificial muscles are exactly what the name implies—manufactured muscles that scientists soon hope to be able to use to perform daily tasks that a natural muscle (i.e., in a human or other mammal) is capable of. It is believed that improved artificial muscles could be used to enhance current prosthetics and micro robots and even to create smart textiles that respond to environmental changes to help make daily tasks easier to perform. Artificial muscles, also sometimes referred to as fiber-based actuators, are thermally and optically controllable, can lift more than hundreds to thousands of times their own weight, and can withstand strains greater than 1000%.

Artificial muscles are currently made out of conventional polymers with negative coefficients of thermal expansion (i.e., polyethylene, nylon) by twist insertion into precursor fibers or by using two-way shape memory polymers, but the efficiency of artificial muscles made out of solely polymers are far from the desired result and capabilities for the muscle. With artificial muscles made only out of conventional polymers or two-way shape memory polymers, a tensile load must be dangled from the artificial muscle to allow the muscle to function. This has significantly limited the usage of artificial muscles because hanging a load is not practical in most applications.

Although several efforts have been made to make free-stranding two-way shape memory polymers, the actuation of these polymers is usually small, the energy output is low, and although one polymer has exhibited muscle behavior under external compression, i.e., beyond free-standing, the actuation is only about 6.2% with a very small external compression (0.05 MPa) under an external compression load of −0.5 N. Therefore, there is a need to make free-standing artificial muscle and muscles beyond free-standing with large actuation and large external compression.

It has been found that the muscle behavior of semicrystalline two-way shape memory polymers persists in two mechanisms. The first is due to the rubber elasticity at temperatures above the crystallization temperature, and the second to the melt/crystallization transition at temperature below crystallization. Unlike metals, polymers change properties over a given temperature range. The individual molecules that make up polymers are very large and have an extended chain-like shape that results in a crystal-like structure under external tension or low temperature or both, which is the origin of expansion of artificial muscles upon cooling. The relatively high levels of elongation that most polymers exhibit without breaking are due in large part to chain structure.

On the other hand, metallic springs can be found in everyday life. Although they are sometimes compared to muscles, they do not exhibit muscle behavior. In fact, metallic springs contract upon cooling and expand upon heating, opposite to the desired mode of action for an artificial muscle. Therefore, how to make conventional metallic springs and other elastic springs into artificial muscles remains a challenge.

Despite advances in artificial muscle research, there is still a scarcity of free-standing actuator systems that have shape memory properties and allow for repeated and/or reversible actuation, which expand with cooling and contract with heating, and which exhibit high efficiency even in the absence of a tensile load. These needs and other needs are satisfied by the present disclosure.

### SUMMARY

In accordance with the purpose(s) of the present disclosure, as embodied and broadly described herein, the disclosure, in one aspect, relates to free-standing artificial muscles having a polymeric core surrounded by an elastic spring such as, for example, a metallic spring The polymeric core can be any two-way shape memory polymer including, but not limited to, a semicrystalline polymer (a polybutadiene polymer, a polycaprolactone polymer, a poly(ethylene-co-vinyl acetate)), a rubber, an ionomer, an elastomer, or a gel. In some aspects, the shape memory polymers can be crosslinked. In an alternative aspect, the polymeric core can be a twisted polymeric fiber. In other aspects, the polymeric core is reprocessable, remoldable, and/or recyclable. In one aspect, the elastic spring can be metal, ceramic, plastic, or any combination thereof. The stiffnesses or spring rate of the spring and polymeric core, the two-way shape memory effect of the polymeric core, and their geometrical dimensions can be optimized to maximize the actuation strain as described herein. Also disclosed are devices incorporating the free-standing artificial muscles.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows measurement of the stiffness of a polybutadiene (PBD) wire.

FIG. 2 shows a PBD-based hybrid muscle clamped by the DMA fixture.

FIG. 3 shows a photograph of polycaprolactone (PCL) based free standing muscle, i.e., a PCL strip is inserted in a conventional metallic spring and clamped by two binder clips at the two ends (left) and a photograph of the PCL based free standing muscle after two-way shape memory test conducted by dynamic mechanical analysis (DMA) (right).

FIG. 4 shows the DMA test results of the effect of tensile stress on two-way shape memory property of the pure PCL sample.

FIG. 5 shows the DMA of two-way shape memory properties of the PCL based free standing muscle under different stress states.

FIGS. 6-8 show strain output relative to temperature and static force for three exemplary artificial PBD-based muscles.

FIG. 9 shows actuation test results for a pure polyethylene artificial muscle.

FIG. 10 shows actuation test results for an artificial muscle including a twisted polyethylene (PE) fiber and commercial compression spring that was 24.6 mm long with a 5.53 mm outer diameter (OD) and 4.75 mm inner diameter (ID). The wire diameter was 0.4 mm. The spring was compressed to 15.24 mm under a compressive load of 3.07 N and the stiffness of the spring was 0.33 N/mm.

FIG. 11 shows actuation test results for an artificial muscle including a twisted polyethylene (PE) fiber and a commercial compression spring that was 25.4 mm long with a 6.10 mm outer diameter (OD) and 4.78 mm inner diameter (ID). The wire diameter was 0.66 mm. The spring was compressed to 7.37 mm under a compressive load of 23.45 N and the stiffness of the spring was 1.30 N/mm.

FIG. 12 shows actuation test results for an artificial muscle including a twisted polyethylene (PE) fiber and a commercial compression spring that was 25.40 mm long with a 7.95 mm outer diameter (OD) and 6.63 mm inner diameter (ID). The wire diameter was 0.66 mm. The spring was compressed to 5.84 mm under a compressive load of 13.66 N and the stiffness of the spring was 0.16 N/mm.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are free-standing artificial muscles containing at least (a) a core comprising a polymer or polymeric material and (b) an elastic spring encasing the core. In an aspect, the polymer can be a two-way shape memory polymer (2W-SMP). In a further aspect, the 2W-SMP can be selected from a semicrystalline 2W-SMP, an elastomeric 2W-SMP, a rubber, an ionomer, a gel, or any combination thereof. In one aspect, the polymeric core can be a polybutadiene polymer, a polycaprolactone polymer, a poly(ethylene-co-vinyl acetate) polymer, a mixed-ester based polymer, or any combination thereof. In another aspect, the core can include a twisted polymeric fiber such as, for example, polyethylene (PE). In another aspect, the polymeric core can be any two-way shape memory polymer known in the art. In one aspect, the artificial muscles are capable of functioning without the need to attach an external weight.

In one aspect, disclosed herein is a hybrid composite muscle which (1) makes two-way shape memory polymer fibers into free-standing muscles, even under external compressive load and (2) transforms elastic springs into artificial muscles. In another aspect, the hybrid muscle is made by inserting pre-tensioned two-way shape memory polymer wire into a pre-compressed spring. In an aspect, different sets of springs with different stiffnesses can be used in the disclosed artificial muscle. In one aspect, the artificial muscles can include a semicrystalline two-way shape memory polymer such as, for example, polybutadiene (PBD). In another aspect, the muscle behavior of the hybrid composite muscles can be investigated using Dynamic Mechanical Analysis (DMA). In a further aspect, in a typical experiment, the strain (response of a system to an applied stress) percentage of the muscle can be measured under different external static forces with increments of ±0.5 N starting from 0 N to ±1 N. Further in this aspect, zero external force means free-standing, while negative force (compression) suggests beyond free-standing. An exemplary muscle exhibited around 12-16% reversible actuation in response to different levels of external static force. In another aspect, another semicrystalline two-way shape memory polymer, such as, for example, polycaprolactone (PCL), can be used to make a free-standing artificial muscle. In still another aspect, a fishing line artificial muscle can be manufactured by twist insertion in a precursor polyethylene (PE) fiber, wherein the PE is used to replace the semicrystalline two-way shape memory polymers in fabricating a free-standing artificial muscle.

In one non-limiting aspect, the polybutadiene polymer can be cis 1,4-polybutadiene, wherein the cis 1,4-polybutadiene has been crosslinked with a crosslinking agent. In another aspect, the crosslinking agent can be dicumyl peroxide.

In another non-limiting aspect, the polycaprolactone polymer has been crosslinked with a crosslinking agent, and wherein, prior to crosslinking, the polycaprolactone polymer has an initial number average molecular weight of from about 30,000 to about 60,000 Da, or of about 30,000; 35,000; 40,000; 45,000; 50,000; 55,000; or about 60,000 Da. In one aspect, the crosslinking agent can be benzoyl peroxide.

In one aspect, the stiffnesses of the spring and polymeric core, the two-way shape memory effect of the polymeric core, and their geometrical dimensions can be optimized to maximize the actuation strain as described herein as described in the Examples. Exemplary, non-limiting dimensions and properties of the spring and polymeric core are described below.

In an aspect, the polymeric core can have a diameter of from about 0.004 to about 2.0 in, or from about 0.014 to about 0.029 in, or of about 0.004, 0.01, 0.014, 0.015, 0.016, 0.017, 0.018, 0.019, 0.02, 0.021, 0.022, 0.023, 0.024, 0.025, 0.026, 0.027, 0.028, 0.029, 0.03, 0.035, 0.04, 0.045, 0.05, 0.055, 0.06, 0.065, 0.07, 0.075, 0.08, 0.085, 0.09, 0.095, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, or about 2 in, or a combination of any of the foregoing values, or a range encompassing any of the foregoing values. In another aspect, the polymeric core can be smaller than 0.004 in or larger than 2.0 in. In any of these aspects, the polymeric core is slightly smaller than the inner diameter of the spring, so that the polymeric core can be inserted into the spring.

In another aspect, the polymeric core can have a stiffness of from about 0.1 lbs/in to about 100,000 lbs in, or from about 3.8 lbs/in to about 17.0 lbs/in, or of about 0.1, 1, 2, 3, 4, 5, 6, 7, 8, 9, 10, 11, 12, 13, 14, 15, 16, 17, 18, 19, 20, 50, 100, 500, 1000, 5000, 10,000, 20,000, 30,000, 40,000, 50,000, 60,000, 70,000, 80,000, 90,000, or about 100,000 lbs/in, or a combination of any of the foregoing values, or a range encompassing any of the foregoing values. In some aspects, the polymeric core can have a stiffness of less than about 3.8 lbs/in or greater than about 17.0 lbs/in.

In any of these aspects, the polymeric core can be flame retardant, reprocessble, and/or recyclable, or any combination thereof.

In one aspect, the elastic spring can be made from a material such as, for example, a metallic material, a ceramic material, a plastic material, or any combination thereof.

In one aspect, when the spring is metallic, the metallic spring can be made from or include steel, stainless steel, zinc-plated stainless steel, cadmium-plated stainless steel, a cobalt-nickel alloy, aluminum, copper, or any combination thereof.

In another aspect, when the spring is ceramic, the ceramic spring can be made from or include beryllium oxide, aluminum nitride, aluminum oxide, boron nitride, fused silica, glass ceramic, mullite, silicon carbide, silicon nitride, zirconium oxide, or any combination thereof.

In still another aspect, when the spring is plastic, the spring can be or include a thermoset polymer such as, for example, epoxy, polyester, polyisocyanurate, or any combination thereof; or can be or include a thermoplastic polymer such as, for example, polyetherimide, polyurethane, polyamide, or any combination thereof; or can be or include a rubbery material such as, for example, styrene-butadiene, nitrile, silicone, or any combination thereof; or can be or include an elastomeric material such as, for example, styrene-butadiene-styrene copolymer, polyester-polyether copolymer, polyamide-polyether, or any combination thereof; or can be or include a fiber reinforced polymer composite such as, for example, carbon fiber reinforced epoxy, glass fiber reinforced polyester, carbon fiber reinforced poly(vinyl ester), or any combination thereof. In one aspect, the plastic spring can include one or more different types of plastic materials listed herein (e.g. a thermoset polymer and a thermoplastic polymer, or a rubbery material and a fiber reinforced polymer composite, or the like).

In still another aspect, the elastic spring has a length of from about 0.01 in to about 10 in, or from 0.3 in to about 0.5 in, or of about 0.01, 0.05, 0.1, 0.15, 0.2, 0.25, 0.3, 0.325, 0.35, 0.375, 0.4, 0.425, 0.45, 0.475, 0.5, 0.75, 1, 2, 3, 4, 5, 6, 7, 8, 9, or about 10 in, or a combination of any of the foregoing values, or a range encompassing any of the foregoing values. In yet another aspect, the elastic spring can have a length of less than about 0.01 in or greater than about 10 in.

In one aspect, the elastic spring has an outer diameter of from about 0.004 in to about 2 in, or from 0.18 in to about 0.36 in, or of about 0.004, 0.01, 0.015, 0.18, 0.2, 0.22, 0.24, 0.26, 0.28, 0.3, 0.32, 0.34, 0.36, 0.5, 0.75, 1, 1.25, 1.5, 1.75, or about 2 in, or a combination of any of the foregoing values, or a range encompassing any of the foregoing values. In another aspect, the elastic spring can have an outer diameter of less than about 0.0.004 in or greater than about 2 in.

In another aspect, the elastic spring has an inner diameter of from about 0.003 in to about 1.90 in, or from 0.152 to about 0.302 in, or of about 0.003, 0.005, 0.01, 0.015, 0.152, 0.16, 0.17, 0.18, 0.19, 0.2, 0.21, 0.22, 0.23, 0.24, 0.25, 0.26, 0.27, 0.28, 0.29, 0.3, 0.302, 0.5, 0.75, 1, 1.25, 1.5, 1.75, or about 1.90 in, or a combination of any of the foregoing values, or a range encompassing any of the foregoing values. In still another aspect, the inner diameter of the elastic spring is slightly larger than the diameter of the polymeric core, enabling the polymeric core to be inserted into the elastic spring. In one aspect, the elastic spring can have an inner diameter of less than about 0.003 in or greater than about 1.90 in.

In one aspect, the elastic spring has a wire diameter of from about 0.001 to about 0.5 in, or from 0.03 to about 0.05 in, or of about 0.001, 0.005, 0.01, 0.03, 0.035, 0.04, 0.041, 0.045, 0.05, 0.075, 0.1, 0.2, 0.3, 0.4, or about 0.5 in, or a combination of any of the foregoing values, or a range encompassing any of the foregoing values. In some aspects, the elastic spring can have a wire diameter of less than about 0.001 in or greater than about 0.5 in.

In some aspects, the elastic spring has a compressed length at maximum load of from about 0.001 to about 2.0 in, or from about 0.5 to about 0.75 in, or of about 0.001, 0.005, 0.1, 0.5, 0.55, 0.6, 0.64, 0.65, 0.7, 0.75, 1, 1.25, 1.5, 1.75, or about 2 in, or a combination of any of the foregoing values, or a range encompassing any of the foregoing values. In still another aspect, the elastic spring can have a compressed length at maximum load of less than about 0.001 in or greater than about 2.0 in.

In some aspects, the elastic spring has a stiffness of from about 3.8 lbs/in to about 17.0 lbs/in, or of about 3.8 lbs/in, 5.9 lbs/in, 8.0 lbs/in, 10.1 lbs/in, 12.2 lb/in, 14.3 lb/in, 16.4 lbs/in, or 17.0 lbs/in, or a combination of any of the foregoing values, or a range encompassing any of the foregoing values. In still another aspect, the elastic spring can have a stiffness less than about 3.8 lbs/in or greater than about 17.0 lbs/in.

In any of these aspects, design and optimization of the artificial muscle, including size, dimension, stiffness, deformation, and other properties of both the polymer core and the spring, can be conducted per the theory presented in Example 4 of this disclosure.

Also disclosed herein are devices including the disclosed artificial muscles. In one aspect, the device can be a soft robot, an aeronautical component, a medical device, another device, or any combination thereof. In one aspect, the medical device can be a prosthesis or artificial limb.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a shape memory polymer” or “an actuator,” includes, but is not limited to, combinations or mixtures of two or more such shape memory polymers or actuators, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

The present disclosure can be described in accordance with the following numbered aspects, which should not be confused with the claims.

Aspect 1. A free-standing artificial muscle comprising:

(a) a core comprising a polymer; and

(b) an elastic spring encasing the core.

Aspect 2. The artificial muscle of aspect 1, wherein the polymer comprises a two-way shape memory polymer (2W-SMP).

Aspect 3. The artificial muscle of aspect 2, wherein the two-way shape memory polymer comprises a semicrystalline 2W-SMP, an elastomeric 2W-SMP, a rubber, an ionomer, a gel, or any combination thereof.

Aspect 4. The artificial muscle of aspect 2 or 3, wherein the two-way shape memory polymer comprises a polybutadiene polymer, a poly(ethylene-co-vinyl acetate) polymer, a polycaprolactone polymer, or any combination thereof.

Aspect 5. The artificial muscle of aspect 4, wherein the polybutadiene polymer comprises cis 1,4-polybutadiene, wherein the cis 1,4-polybutadiene has been crosslinked with a crosslinking agent.

Aspect 6. The artificial muscle of aspect 4, wherein the polycaprolactone polymer has been crosslinked with a crosslinking agent, and wherein, prior to crosslinking, the polycaprolactone polymer has an initial number average molecular weight of from about 30,000 Da to about 60,000 Da.

Aspect 7. The artificial muscle of aspect 4, wherein the poly(ethylene-co-vinyl acetate) polymer has been crosslinked with a crosslinking agent.

Aspect 8. The artificial muscle of any one of aspects 5-7, wherein the crosslinking agent comprises benzoyl peroxide, dicumyl peroxide, lauroyl peroxide (LPO), or any combination thereof.

Aspect 9. The artificial muscle of aspect 1, wherein the core comprises a twisted polymeric fiber.

Aspect 10. The artificial muscle of aspect 9, wherein the twisted polymeric fiber comprises polyethylene (PE).

Aspect 11. The artificial muscle of aspect 9, wherein the twisted polymeric fiber comprises nylon.

Aspect 12. The artificial muscle of aspect 9, wherein the twisted polymeric fiber comprises one or more two-way shape memory polymers.

Aspect 13. The artificial muscle of any one of aspects 1-8, wherein the core has a diameter of from about 0.004 to about 2.0 in.

Aspect 14. The artificial muscle of aspect 1, wherein the core has a stiffness of from about 0.1 lbs/in to about 100,000 lbs/in.

Aspect 15. The artificial muscle of any one of aspects 1-14, wherein the elastic spring comprises a metal, a ceramic, a plastic, or any combination thereof.

Aspect 16. The artificial muscle of aspect 15, wherein the metal comprises steel, stainless steel, zinc-plated stainless steel, cadmium-plated stainless steel, a cobalt-nickel alloy, aluminum, copper, or any combination thereof.

Aspect 17. The artificial muscle of aspect 15, wherein the ceramic comprises beryllium oxide, aluminum nitride, aluminum oxide, boron nitride, fused silica, glass ceramic, mullite, silicon carbide, silicon nitride, zirconium oxide, or any combination thereof.

Aspect 18. The artificial muscle of aspect 15, wherein the plastic comprises a thermoset polymer, a thermoplastic polymer, a rubbery material, an elastomeric material, a fiber reinforced polymer composite, or any combination thereof.

Aspect 19. The artificial muscle of aspect 18, wherein the thermoset polymer comprises epoxy, polyester, polyisocyanurate, or any combination thereof.

Aspect 20. The artificial muscle of aspect 18, wherein thermoplastic polymer comprises polyetherimide, polyurethane, polyamide, or any combination thereof.

Aspect 21. The artificial muscle of aspect 18, wherein the rubbery material comprises styrene-butadiene, nitrile, silicone, or any combination thereof.

Aspect 22. The artificial muscle of aspect 18, wherein the elastomeric material comprises styrene-butadiene-styrene copolymer, polyester-polyether copolymer, polyamide-polyether, or any combination thereof.

Aspect 23. The artificial muscle of aspect 18, wherein the fiber reinforced polymer composite comprises carbon fiber reinforced epoxy, glass fiber reinforced polyester, carbon fiber reinforced poly(vinyl ester), or any combination thereof.

Aspect 24. The artificial muscle of any one of aspects 1-23, wherein the elastic spring has a length of from about 0.01 in to about 10.0 in.

Aspect 25. The artificial muscle of any one of aspects 1-24, wherein the elastic spring has a length of about 0.375 in.

Aspect 26. The artificial muscle of any one of aspects 1-25, wherein the elastic spring has an outer diameter of from about 0.004 in to about 2.0 in.

Aspect 27. The artificial muscle of any one of aspects 1-26, wherein the elastic spring has an inner diameter of from about 0.003 in to about 1.90 in.

Aspect 28. The artificial muscle of any one of aspects 1-27, wherein the elastic spring has a wire diameter of from about 0.001 to about 0.5 in.

Aspect 29. The artificial muscle of any one of aspects 1-28, wherein the elastic spring has a wire diameter of about 0.041 in.

Aspect 30. The artificial muscle of any one of aspects 1-29, wherein the elastic spring has a compressed length at maximum load of from about 0.001 to about 2.0 in.

Aspect 31. The artificial muscle of any one of aspects 1-30, wherein the elastic spring has a compressed length at maximum load of about 0.64 in.

Aspect 32. The artificial muscle of any one of aspects 1-31, wherein the core is flame retardant.

Aspect 33. The artificial muscle of any one of aspects 1-32, wherein the core is reprocessable.

Aspect 34. The artificial muscle of any one of aspects 1-33, wherein the core is recyclable.

Aspect 35. A device comprising the artificial muscle of any one of aspects 1-34.

Aspect 36. The device of aspect 35, wherein the device comprises a component of soft robot, an aeronautical component, a medical device, or any combination thereof.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in °C. or is at ambient temperature, and pressure is at or near atmospheric.

### Example 1: Free-Standing Artificial Muscle with Two-Way Shape Memory Polycaprolactone

### Preparation of Two-Way Shape Memory Polycaprolactone (PCL)

PCL with an average Mₙ 45,000, benzoyl peroxide (BPO) as initiator, and tetrahydrofuran (THF) as solvent were purchased from Sigma-Aldrich and used without further purification. 18 g PCL powder was dissolved in 50 mL THF by mechanically stirring at room temperature for 6 hours. 2 g initiator BPO was then added into the solution and mixed well. The mixture was then poured onto a polytetrafluoroethylene (PTFE) sheet and placed in a hood to allow air dry for 1 day. The obtained PCL was further dried at room temperature in a vacuum oven for 6 hours to completely remove the solvent THF. The fully dried PCL was cut into small pieces and put into a PTFE mold to crosslink at 130° C. for 20 min under pressure of 20 MPa using a hot press machine. The thickness of the obtained PCL sheet was around 1 mm. Slender PCL bars with a size of 30×3×1mm were cut from the PCL sheet for the two-way shape memory test.

### Preparation of PCL-Based Free-Standing Muscle

A compression metallic spring with 1 in long, 0.343 in OD (outer diameter), and 0.262 in ID (inner diameter) was ordered from McMaster-Carr. The wire diameter was 0.041 in, compressed length at maximum load was 0.64 in, maximum load was 6.65 lbs., and compression stiffness was 8.43 lbs./in. To prepare the PCL based free standing muscle, a slender cuboid PCL bar prepared previously was inserted into the compression spring. The spring was then compressed, and both ends of the PCL bar were clamped by clips, forming a composite stand- alone spring. When conducting two-way shape memory test by dynamic mechanical analysis (DMA), the spring was firstly manually compressed, and both ends of the PCL bar were then clamped by the DMA built-in clips. The length of the compressed composite spring was about 8.3 mm. The temperature range for the two-way shape memory test was from −10 to 70° C. and the heating/cooling rate was 5° C/min. When reaching to the highest/lowest temperature point, the sample was iso-thermalized for 5 min. The tensile stress or compressive stress was applied after 5 min isothermal at −10° C. when necessary.

### Results and Discussion

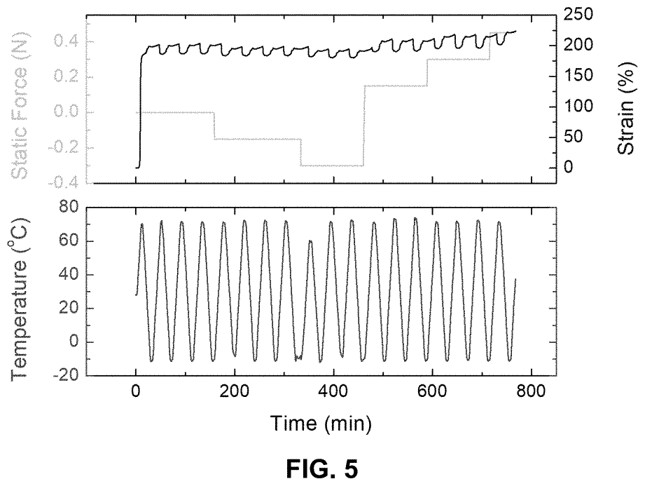

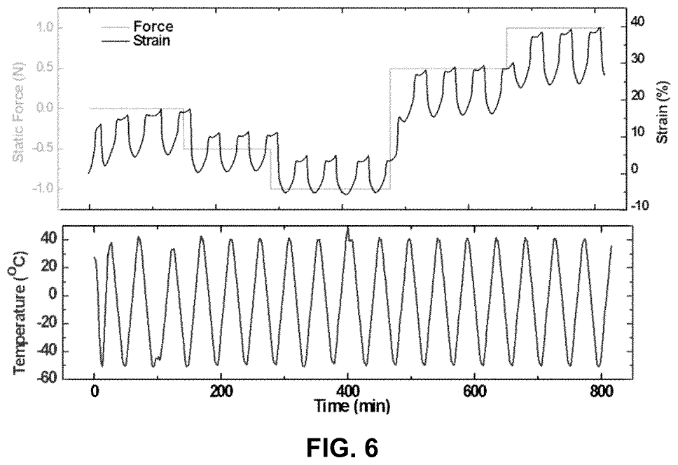

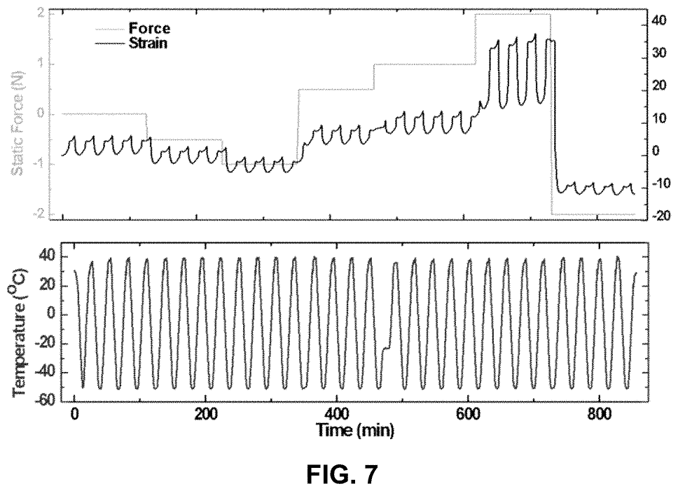

FIG. 3 shows the composite spring with the PCL bar clamped by clip (left) and by DMA fixture (right). FIG. 4 shows the two-way shape memory effect (2W-SME) of the pure PCL sample. It is seen that, under a constant tensile load, the system expands upon cooling, and contracts upon heating. For example, under 0.2 MPa of external tensile stress, the PCL sample expands about 70% when the temperature drops from 70° C. to −10° C. Again, a typical two-mechanism controlled 2W-SME is seen: at higher temperature, it is caused by rubber elasticity, and at lower temperature, it is caused by the melting/crystallization transition. FIG. 5 shows the 2W-SME or artificial muscle behavior of the hybrid composite muscle. The PCL based composite muscle shows significant 2W-SME even under an external compression load. Very few known polymers possess this type of 2W-SME, and of those that do, the actuation strain is typically so small as to be negligible. However, here, it is shown that under 0.1 MPa external compressive force, the hybrid muscle expands by about 14% when the temperature drops from 70° C. to −10° C.

Therefore, from FIG. 5, it has been shown that a conventional metallic spring can be transformed into an artificial muscle. The resulting artificial muscle not only is stand-alone (without external load and only by changing temperature), but also exhibits muscle behavior even under external compressive load.

In summary, this system transforms a conventional metallic spring to an artificial muscle; and additionally transforms existing 2W-SMPs not only to stand-alone muscle (without any external load), but also muscle under external compression, i.e., true artificial muscle.

### Example 2: Free-Standing Polybutadiene (PBD) Artificial Muscle

### Materials and Methods

Cis poly(1, 4-butadiene) (PBD) under trade name Budene 1208 from Goodyear Chemical (Akron, Ohio, USA) was used as the reagent. The viscosity is 40 (Mooney ML 1+4 at 100° C.). The onset glass transition temperature is −104° C. The solvent chloroform from Sigma-Aldrich (St. Louis, Mo., USA), was used to dissolve cis polybutadiene. A sticky homogeneous solution was acquired after overnight stirring. The ratio of PBD to chloroform is roughly 1 to 10. The curing agent, dicumyl peroxide (DCP), from Sigma-Aldrich, at the amount of 3 wt % of cis polybutadiene, was added into the solution. After thorough mixing of the dicumyl peroxide with cis polybutadiene solution, chloroform was removed by simple evaporation overnight in a hood at ambient temperature. The dried cis polybutadiene and dicumyl peroxide mixture was then clamped in a Teflon mold and cured for 30 min in a 150° C. oven. The crosslinked cis polybutadiene (cPBD) in a semitransparent color was yielded. The sheet was wrapped and rubbed into a cylinder for use. The stiffness of the 4.36 mm diameter PBD wire, as determined based Hooke's law per a spring balance, is found to be 0.127 N/mm.

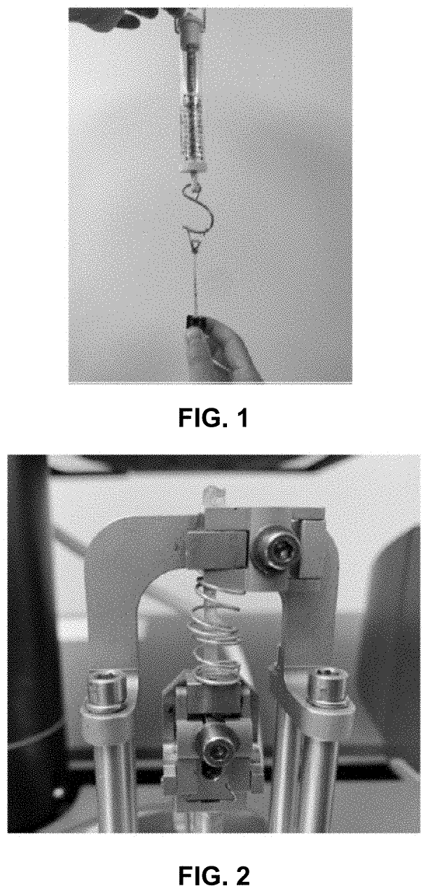

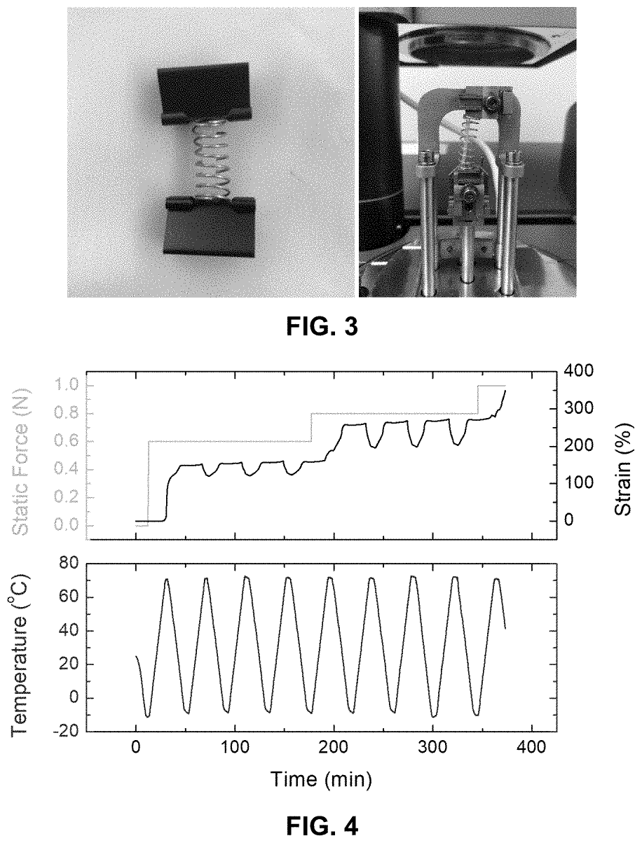

Two short springs were aligned and fully compressed while holding the springs together with tweezers before using copper wire to tie the system together to secure it such that the springs do not come apart. Using scissors, the PBD cylinder was cut into a strip with dimensions 18.58 mm as the length and 4.36 mm as the diameter, and then inserted into the compressed spring. One end of the PBD wire was inserted into the upper tension clamp of the dynamic mechanical analysis (DMA) instrument and clamped. Afterwards, the opposite end of the PBD wire was stretched to 200 percent of its original length and inserted and fixed into the lower tension clamp of the DMA instrument. Once the PBD wire was secured, the copper wire was cut, securing the springs and obtaining a hybrid artificial muscle. Three sets of springs are used, leading to three different muscles. FIG. 1 shows measuring the stiffness of the PBD wire and FIG. 2 shows the hybrid muscle is clamped by the DMA fixture.

Using software provided with a dynamic mechanical analysis (DMA) instrument (model Q800 DMA from TA Instruments, DE, USA) the testing setup was programmed, including the heating and cooling rate (10° C/min), the lowest temperature (−50° C.) and the highest temperature (+40° C.), and the constant load (−1 N, −0.5 N, 0 N, +0.5 N, +1 N) (here, the negative sign represents compressive load and positive sign means tensile load; 0 N load represents free-standing and negative load represents muscle beyond free-standing). Within each constant load, the muscle was set to perform three cycles of heating and cooling to trigger muscle action and to observe any possible creep. The DMA instrument recorded the load or stress, displacement or strain, and temperature change with respect to time, with the process being repeated for each artificial muscle sample.

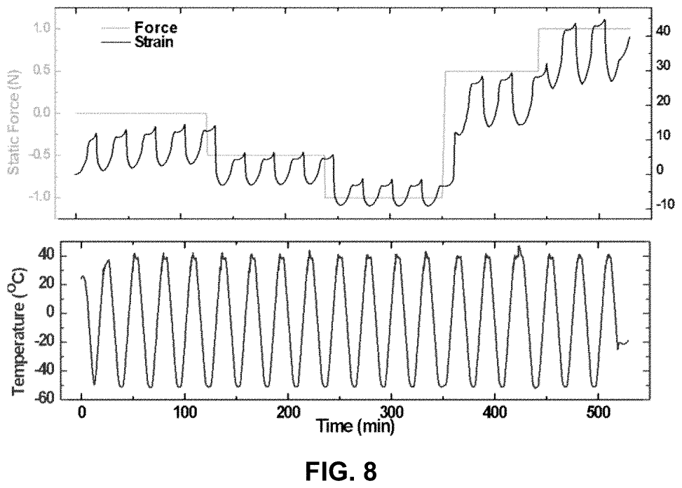

As shown in Table 1, one PBD wire and three springs were chosen for validation experiments. The actuations of the three hybrid muscles are shown in FIGS. 6-8, respectively. Each hybrid muscle was subjected to three types of loads: under tension (conventional muscle), under zero external load (free-standing muscle), and under compression (beyond free-standing).

TABLE 1
PBD Spring Measurements

| | | | | Diameter | Stiffness |
|---|---|---|---|---|---|
| | | Outer | Inner | of | of |
| Spring | Length | Diameter | Diameter | PBD Wire | Spring |
| Identifier | (in) | (in) | (in) | (in) | (lbs./in) |
| Spring 01 | 0.375 | 0.240 | 0.204 | 0.018 | 5.9 |
| Spring 02 | 0.375 | 0.360 | 0.302 | 0.029 | 17.0 |
| Spring 03 | 0.375 | 0.180 | 0.152 | 0.014 | 3.8 |

### Analysis and Discussion

All muscles exhibited similar patterns throughout all test runs at varying static force loads. As the temperature within the DMA reached −50° C., a maximum output in strain was observed (expansion), and when the temperature reached 40° C., there was a minimum output in strain, leading to contraction.

Observing the pattern at the crest of the strain graph on the cooling branch where there was a peak followed by a dip, which was then followed by the “true maximum” of the graph, with similar results for all testing of all muscles. At temperatures above the crystallization temperature, the expansion upon cooling is driven by rubber elasticity, while at temperatures below the crystallization temperature, the muscle behavior is driven by the melting/crystallization transition.

Muscle 1: Within the differences in the amount of applied static force on the polymer in FIG. 6, since the static force applied was controlled in increments of ±0.5 N, it was expected that a similar level of maximum strain percentage moving through the applied forces would be observed. However, this was not the case; between 0 N and −0.5 N, there was a −5% strain difference at the peak, whereas between 0 N and +0.5 N, there was a +13% strain difference at the peak. However, as static force increased from +0.5 N to 1 N, there was a common +13% strain difference at the peak, and as static force decreased from −0.5 N to −1 N, there was a common −5% strain difference at the peak. Going in either the positive or negative direction of applied force, the amount of strain percentage is the same between increments of the same sign, but there is a differing result when comparing positive and negative signs.

A possible explanation is that, under external compression, the PBD wire within the assembly is shortened, which is equivalent to reduction in the pre-tension on the PBD. It is known that a smaller pre-tension usually leads to a small expansion upon cooling. This same argument can be used for the case under external tensile load, i.e., the actuation increases as the external tensile load increases.

Muscle 2: As seen in FIG. 7, muscle two, or the muscle with the highest stiffness exhibited a lower strain percentage output in comparison to muscle 1. These results indicate the artificial muscle was much more consistent in maintaining similar levels of strain throughout different static forces, with the percentage always hovering around 8%. This muscle was also subjected to additional static forces of +2 N and −2 N. At −2 N, the strain decreased to roughly around 5%, most likely due to the spring's stiffness not allowing it to perform such elongation action. However, at +2 N, the strain was tremendously increased. From the normal strain of around 8%, at this stage, the muscle exhibited over 20% strain output. Thus, muscle 2 is able to function extremely well under tension.

Out of the three muscles, muscle 2 exhibited the lowest amount of creep, which is highly desirable. The creep on this polymer system is nearly non-existent. This shows that higher stiffness leads to a more stable actuation and decrease of creep alongside other characteristics such as the various spring diameters as seen in Table 1. The creep for muscle 1 was observed as the cycles progressed at about +0.5%. In addition, creep cannot be eliminated, only reduced to a minimum, such that the polymer can still proceed with its purpose.

Muscle 3: Regarding muscle 3, this muscle was the least stiff, seen in Table 1. After running muscle 2, it was already expected for muscle 3 with a lower stiffness to have a greater actuation strain output and the largest creep out of the three muscles. This indeed was observed as indicated by the results in FIG. 8. The strain varied from roughly 9% at −1 N static force to 17% at +1 N static force. The creep in the graph was roughly 0.5% at 0 N, 0% at −0.5 N and −1 N, but almost reaching 1% at +0.5 N and +1 N.

Comparison with the pure PBD: A pure PBD developed by Lu et al. is the only one that demonstrated muscle behavior beyond free-standing. Based on these results, the PBD exhibited 6.2% expansion when it is subjected to −0.5 N compressive load. For Muscle 2, it shows about 5% expansion when it is subjected to 2 N of compressive load. One thing worth noting is that, Lu et al. used the original length of the PBD fiber to calculate the actuation, while experiments herein used the stretched length to calculate the extension. During the assembling of the hybrid muscles, the PBD fiber was stretched to about 200% of its initial length. If using the original length of the PBD fiber to calculate the actuation strain, this value should be multiplied by 3, which yields 5%×3=15% extension upon cooling. Therefore, as compared to the pure PBD, the hybrid muscle can sustain higher external compression (2 N versus 0.5 N) and produce higher actuation (15% versus 6.2%). Hence, the disclosed hybrid muscle is the only known artificial muscle that exhibits superior muscle behavior beyond free-standing.

### Conclusion

These experiments focused around artificial muscles and creating alterations to existing artificial muscles to make them more effective at actuation and increasing their versatility by attempting to create a “free standing” or even “beyond free-standing” artificial muscle. The experiments presented thus far indicate that the free standing artificial muscle constructed using two-way shape memory polybutadiene and conventional metallic springs was successful at performing “beyond free-standing” behaviors. Instead of expanding when heated, the muscle shrinks when heated, and instead of shrinking when cooled, it expands. Previously, this type of muscle was not known to exist and the only known artificial muscles were ones that required a hanging weight to enable actuation. Not only does the disclosed muscle have the ability to actuate without tensile load, but it can also actuate under compression load. Therefore, this hybrid muscle is not only a free standing muscle, but is also beyond free standing.

By combining polybutadiene and conventional metallic springs, static force and strain tests were run on the artificial muscle. The static forces were placed at 0.5 N increments until −1 N or −2 N and 1 N of static force was achieved. Then, the muscle was heated and cooled over a temperature range from 40° C. to −50° C. in the DMA instrument. It was observed in the DMA software provided with the instrument that muscle 1 exhibited a strain percentage of roughly 12-16% per cycle for muscle 1, 5% to 20% for muscle 2, and 9-17% for muscle 3 across different static forces, proving that the compression and expansion of the muscle is large enough to be seen by the naked eye.

Because three sets of springs were used and different levels of external load are applied, it can be seen that the spring stiffness has a significant effect on the muscle behavior. For the case of free-standing, muscle 1 has the highest and muscle 2 has the lowest actuation; for case of beyond free-standing, i.e., under external compressive load, muscle 3 is the best, and the least is muscle 2; for the muscle under external tensile load, muscle 3 has the largest actuation. Therefore, an appropriate combination of stiffness of the spring and PBD, pre-strain, cycling temperature, and external load can optimize this type of muscle design.

### Example 3: Free-Standing Artificial Muscle Made of Hybrid Fishing Line and Metallic Spring

A further experiment showed that the two-way shape memory polymer can be replaced by other polymeric materials to fabricate hybrid free-standing artificial muscle. In this proof-of-concept study, a commercial fishing line, polyethylene (PE), was made into polymeric artificial muscle by twist-insertion. Commercial PE fishing line with a diameter of 533 μm (ZEBCO OMNIFLEX3OLBA) was used to fabricate polymer artificial muscles. The PE copolymer monofilament fishing line was twisted using a commercial reversible rotor with the bottom end of the fishing line fixed while the upper end was attached to the rotor. The bottom end was loaded with 360 grams of load, which is 16 MPa when normalized to the PE line cross-sectional area. The PE line was twisted in a counterclockwise direction at room temperature using a manually controlled rotor. The twisting number for coiling is determined by the effective length of fishing line from the bottom end to the upper end. It is found that the slope between the twisting number and effective fiber length is 0.43 twists/mm, which is a critical value for the PE fiber from twist to coil. For example, with an effective length of 100 mm, the critical number of twisting is 43. When the value is larger than 43, it begins to coil; otherwise, it needs more twists. Prior to being coiled, a steel wire with a diameter of 0.75 mm was used as mandrel and wrapped with the twisted PE fiber in the counterclockwise direction. In order to keep the bias angle constant, the wrapping rate must be the same as the twisting rate. The coiled configuration was maintained by fixing both muscle ends once the fiber is coiled completely. It was then put into an oven and maintained for at least 95 min under 80° C. for annealing. The mandrel was removed after being cooled down to room temperature. After annealing, the PE artificial muscle was obtained with a spring index of C =2.4. After twist insertion and annealing, the precursor fiber, i.e., fishing line, behaves like two-way shape memory polymer, i.e., expansion upon cooling, and contraction upon heating, again, under a constant tensile load. The fabrication process for the hybrid muscle follows the same procedure as discussed previously. The only change is to replace the two-way shape memory polymer bar with a piece of fishing line artificial muscle.

In this study, three type of metallic springs were used. The temperature range for two-way shape memory effect test was 50˜100° C., and the heating/cooling rate was 5° C./min. When reaching to the highest/lowest temperature point, the sample was iso-thermalized for 5 min. The tensile stress or compressive stress was applied after a 5 min isothermal hold at 50° C. based on the designed loading pattern and loading sequence.

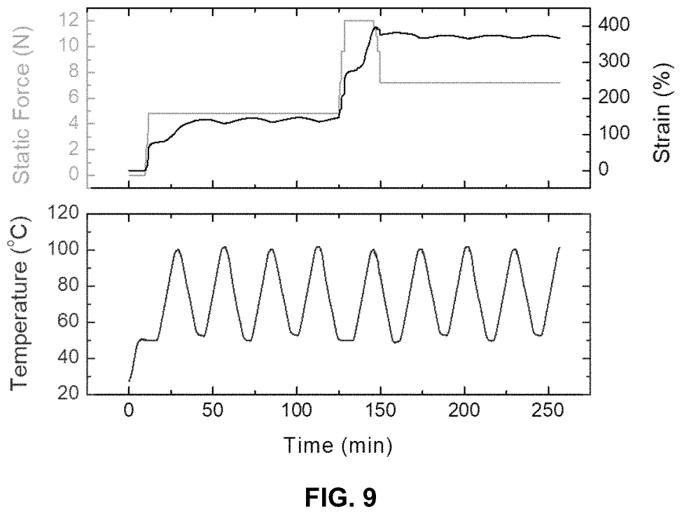

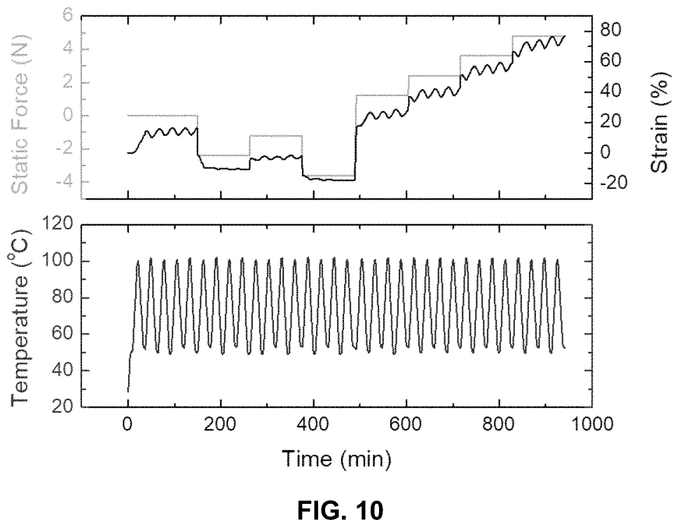

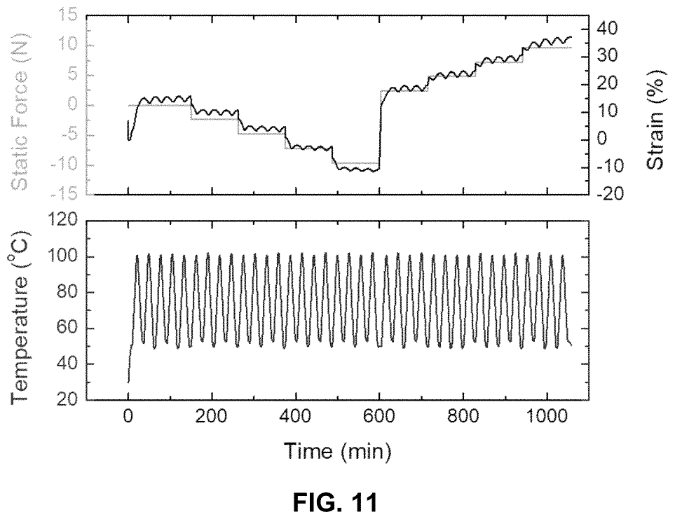

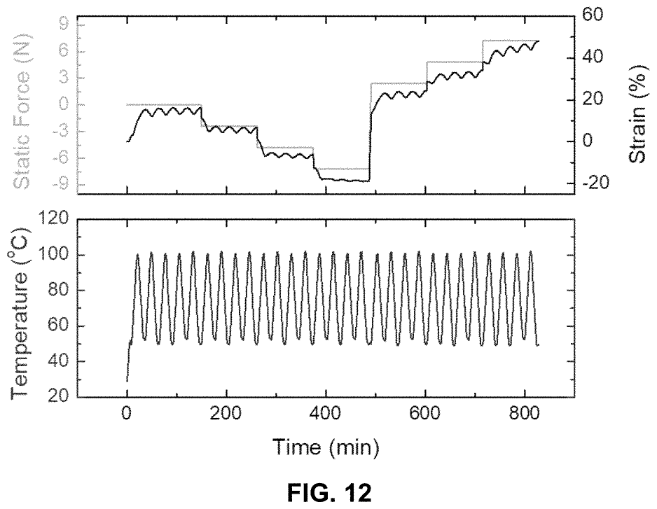

FIG. 9 shows the behavior of the pure fishing line artificial muscle. FIGS. 10-12 show the actuation test results of the hybrid artificial muscle by the same DMA machine. It is seen that the pure fishing line artificial muscle exhibits two-way shape memory effect under an external tensile load. For the hybrid free-standing artificial muscles, they demonstrate two-way shape memory effect either under an external tensile load, zero external load, or external compressive load. This again validates that the hybrid muscle is free-standing and beyond free-standing. It is also seen that, with the highest stiffness of the metallic spring (FIG. 11), the hybrid artificial muscle shows higher actuation strain even under higher external compressive load. It is also found that, the two-way actuation strain of the hybrid artificial muscles is the highest under external tensile load, followed by zero external load, and the lowest is under external compression load, which is in agreement with the theoretical model in Example 3.

### Example 4: Theory

This section provides theoretical models for the hybrid muscle in order to optimize the artificial muscle design. The following assumptions are made. (1) Both the metallic spring and the two-way shape memory polymer (2W-SMP) fiber are linear elastic materials and obey the Hooke's law. (2) The spring will be pre-compressed to its maximum deformation, i.e., the pitch becomes almost zero. (3) After fabrication, the spring is in compression and the 2W-SMP wire is in tension. (4) The stiffness of the spring and 2W-SMP is independent of the deformation, i.e., constant. (5) Compression and compressive force are negative and tension and tensile force are positive. (6) While the stiffness of the 2W-SMP depends on temperature, the stiffness of the spring is independent of temperature in the range of actuation temperature considered. (7) Deformation or strain is measured with respect to the original length of the spring or the 2W-SMP cylinder.

In the examples and equations below, the original length, prestrain immediately before attachment, strain during the process after attachment until the assembly or muscle is in equilibrium, total strain after fabrication, and the stiffness are L₁, L₂, λ₁₁, λ₂₁, λ₁₂, λ₂₂,ψ₁, ψ₂, K₁, and K₂ for the 2W-SMP and spring, respectively. The spring and the 2W-SMP cylinder have the same length immediately before assembling them together:

L₁(1+λ₁₁)=L₂(1+λ₂₁) (1)

The displacement or deformation of the spring and 2W-SMP from immediately after hooking them together to equilibrium must be equal:

L₁λ₁₂=L₂λ₂₂ (2)

Force equilibrium requires:

L₁K₁(λ₁₁+λ₁₂)=−L₂K₂(λ₂₁+λ₂₂) (3 )

Solving the simultaneous equations gives the following result:

$$\begin{matrix}{λ}_{12} = - \frac{{K}_{11} {λ}_{11} ( 1 + {λ}_{21} ) + {K}_{2} {λ}_{21} ( 1 + {λ}_{11} )}{( {K}_{1} + {K}_{2} ) ( 1 + {λ}_{21} )} & ( 4 ) \\ {λ}_{22} = - \frac{{K}_{1} {λ}_{11} ( 1 + [ {0}_{21} ) + {K}_{2} {λ}_{21} ( 1 + {λ}_{11} )}{( {K}_{1} + {K}_{2} ) ( 1 + {λ}_{11} )} & ( 5 )\end{matrix}$$

The strain after assembling the hybrid muscle is:

$$\begin{matrix}{ψ}_{1} = \frac{{K}_{2} ( {λ}_{11} - {λ}_{21} )}{( {K}_{1} + {K}_{2} ) ( 1 + {λ}_{21} )} & ( 6 ) \\ {ψ}_{2} = \frac{{K}_{1} ( {λ}_{21} - {λ}_{11} )}{( {K}_{1} + {K}_{2} ) ( 1 + {λ}_{11} )} & ( 7 )\end{matrix}$$

Because λ₁₁>0 and λ₂₁<0, it is clear that after assembling the hybrid muscle, the PBD is in tension (ψ₁>0) and the spring is in compression (ψ₂<0). Now the hybrid spring is ready for actuation according to one of the following three cases.

### Case 1: Free-Standing Muscle (With Zero External Load Under Temperature Cycling)

In this case, there is no external load. The muscle actuates by simply cycling the temperatures. To solve the actuation strain of the muscle, why and how the muscle actuates must be determined. When temperature drops, the 2W-SMP first experiences expansion due to rubber elasticity, followed by the melting/crystallization transition. This can be clearly seen from the actuation tests of the pure PCL in FIG. 4. Under a constant external load, the 2W-SMP expands as temperature drops. This is impossible for conventional polymers because as temperature drops, the polymer hardens and under the same external load, the polymer must contract rather than expand. Therefore, the self-expansion can be treated as “softening” of the 2W-SMP and equivalent stiffness can be used to represent this softening behavior. Clearly, the equivalent stiffness can be found by using the load divided by the actuation during the actuation test of the pure 2W-SM P. In other words, the temperature induced actuation is treated as an isothermal event; in this event, the 2W-SMP softens. Assuming the equivalent stiffness of the 2W-SMP is K_{eq}, the following relationship applies:

$${K}_{e q} = \frac{F}{D} \qquad ( 8 )$$

where F is the applied tensile load during actuation test of the 2W-SMP, and D is the actuation strain or stretch of the 2W-SMP during the actuation test. Obviously, D depends on the actuation temperature range.

During temperature induced actuation of the hybrid muscle, the 2W-SMP and the spring must actuate the same length, and must keep force equilibrium:

L₁λ₁₃=L₂λ₂₃ (9)

K_{eq}L₁(λ₁₁+λ₁₂+λ₁₃)=−K₂L₂(λ₂₁+λ₂₂+λ₂₃) (10 )

where λ₁₃ and λ₂₃ are the actuation strain of the 2W-SMP and spring, respectively.

Using Equations 1, 2, 4, 5, 9, and 10, the actuation strain can be solved as:

$$\begin{matrix}{λ}_{13} = \frac{{K}_{2} ( {K}_{1} - {K}_{eq} ) ( {λ}_{11} - {λ}_{21} )}{( {K}_{eq} + {K}_{2} ) ( {K}_{1} + {K}_{2} ) ( 1 + {λ}_{21} )} & ( 11 ) \\ {λ}_{23} = \frac{{K}_{2} ( {K}_{1} - {K}_{eq} ) ( {λ}_{11} - {λ}_{21} )}{( {K}_{eq} + {K}_{2} ) ( {K}_{1} + {K}_{2} ) ( 1 + {λ}_{11} )} & ( 12 )\end{matrix}$$

Because K_{eq}<K₁, λ₁₁>0, and −1<λ₂₁<0, it is obvious that both λ₁₃ and λ₂₃ are positive, i.e., they expand when temperature drops or behave like a muscle.

The purpose for artificial muscle is to maximize λ₁₃ and λ₂₃. It is seen from Eqs. (11) and (12) that it is a multi-variable function. It depends on the stiffness of the spring and 2W-SMP at muscle assembling temperature (usually room temperature), the pre-strain before connection of the two parts, and the equivalent stiffness of the “softened” 2W-SMP, which depends on the applied tensile load and tensile actuation capability of the pure 2W-SMP, as demonstrated in Eq. (8)

### Case 2: The Hybrid Muscle is Subjected to a Constant External Tensile Load

The effect of the tensile load is to create additional tensile strain in the 2W-SMP, and reduce the compressive prestrain in the spring.

Assuming the external load F_{T} is applied at temperature T, and the stiffness of the 2W-SMP is thus K_{1T}, the additional strain can be calculated in the 2W-SMP and spring as λ₁₄ and λ₂₄:

F_{T}=K_{1T}L₁λ₁₄+K₂L₂λ₂₄ (13)

L₁λ₁₄=L₂λ₂₄ (14)

Solving the simultaneous Eqs. (13) and (14), the following solution is obtained:

$${λ}_{14} = \frac{{F}_{T}}{( {K}_{1 T} + {K}_{2} ) {L}_{1}} \qquad ( 15 )$$

$${λ}_{24} = \frac{{F}_{T}}{( {K}_{1 T} + {K}_{2} ) {L}_{2}} \qquad ( 16 )$$

In Eqs. (11) and (12), λ₁₁ should be replaced by (λ₁₁+λ₁₄) and λ₂₁ by (λ₂₁+λ₂₄) in order to calculate the actuation upon cooling. Because λ₁₄ and λ₂₄ are positive, they tend to increase the muscle actuation as compared to Case 1.

### Case 3: The Hybrid Muscle is Subjected to External Compressive Load

Assuming the external load if Fc, which is negative, the additional prestrain in the 2W-SMP (λ₁₅) and spring (λ₂₅) can be determined using a similar equation, obtaining:

$${λ}_{15} = \frac{{F}_{C}}{( {K}_{1 T} + {K}_{2} ) {L}_{1}} \qquad ( 17 )$$

$${λ}_{25} = \frac{{F}_{T C}}{( {K}_{1 T} + {K}_{2} ) {L}_{2}} \qquad ( 18 )$$

Again, in Eqs. (11) and (12), λ₁₁ should be replaced by (λ₁₁+λ₁₅) and λ₂₁ by (λ₂₁+λ₂₅) in order to calculate the actuation upon cooling. Because λ₁₅ and λ₂₅ are negative, they tend to decrease the muscle actuation as compared to Case 1.

### REFERENCES

1. T. Mirfakhrai, et al. “Polymer Artificial Muscles.” Materials Today, Vol. 10, No. 44, pp. 30-38, (2007).

2. C. S. Haines , et al. “Artificial Muscles from Fishing Line and Sewing”. Science, Vol. 343, No. 6173, pp. 868-872, (2014).

3. J. Fan et al. “High performance and tunable artificial muscle based on two-way shape memory polymer”. RSC Advances, Vol. 7, No. 2, pp. 1127-1136, (2017).

4. L. Lu, et al. “Giant Reversible Elongation upon Cooling and Contraction upon Heating for a Crosslinked Cis Poly(1, 4-Butadiene) System at Temperatures below Zero Celsius. ” Scientific Reports, Vol. 8, paper number 14233, (2018).

5. C. Yan, et al “A phenomenological constitutive model for semicrystalline two-way shape memory polymers. ” International Journal of Mechanical Sciences, Vol. 177, paper number 105552, (July 2020).

What is claimed is:

1. A free-standing artificial muscle comprising:
(a) a core comprising a polymer; and
(b) an elastic spring encasing the core.

2. The artificial muscle of claim 1, wherein the polymer has been crosslinked with a crosslinking agent.

3. The artificial muscle of claim 2, wherein the crosslinking agent comprises benzoyl peroxide, dicumyl peroxide, lauroyl peroxide (LPO), or any combination thereof.

4. The artificial muscle of claim 1, wherein the polymer comprises a two-way shape memory polymer (2W-SMP).

5. The artificial muscle of claim 4, wherein the two-way shape memory polymer comprises a semicrystalline 2W-SMP, an elastomeric 2W-SMP, a rubber, an ionomer, a gel, or any combination thereof.

6. The artificial muscle of claim 4, wherein the two-way shape memory polymer comprises a polybutadiene polymer, a poly(ethylene-co-vinyl acetate) polymer, a polycaprolactone polymer, or any combination thereof.

7. The artificial muscle of claim 6, wherein the polycaprolactone polymer has an initial number average molecular weight of from about 30,000 to about 60,000 Da.

8. The artificial muscle of claim 1, wherein the core comprises a twisted polymeric fiber.

9. The artificial muscle of claim 8, wherein the twisted polymeric fiber comprises polyethylene (PE), nylon, or any combination thereof.

10. The artificial muscle of claim 1, wherein the core has a diameter of from about 0.004 in to about 2.0 in.

11. The artificial muscle of claim 1, wherein the core has a stiffness of from about 0.1 lbs/in to about 100,000 lbs/in.

12. The artificial muscle of claim 1, wherein the elastic spring comprises a metal, a ceramic, a plastic, or any combination thereof.

13. The artificial muscle of claim 1, wherein the elastic spring has a length of from about 0.01 in to about 10.0 in.

14. The artificial muscle of claim 1, wherein the elastic spring has an outer diameter of from about 0.004 in to about 2.0 in.

15. The artificial muscle of claim 1, wherein the elastic spring has an inner diameter of from about 0.003 in to about 1.90 in.

16. The artificial muscle of claim 1, wherein the elastic spring has a wire diameter of from about 0.001 to about 0.5 in.

17. The artificial muscle of claim 1, wherein the elastic spring has a compressed length at maximum load of from about 0.001 to about 2.0 in.

18. The artificial muscle of claim 1, wherein the core is recyclable.

19. A device comprising the artificial muscle of claim 1.

20. The device of claim 19, wherein the device comprises a component of soft robot, an aeronautical component, a medical device, or any combination thereof.
